(19) United States
(12) Patent Application Publication
(10) Pub. No.: US 2023/0233811 A1
(43) Pub. Date: Jul. 27, 2023

(54) CATHETER SYSTEM HAVING A BLOOD SHIELD

(71) Applicant: Becton, Dickinson and Company, Franklin Lakes, NJ (US)

(72) Inventors: Balaji Kannan, Chennai (IN); Sakthivel Karthikeyan, Bangalore (IN); Deepan Vikash, Tamil Nadu (IN)

(21) Appl. No.: 18/094,763

(22) Filed: Jan. 9, 2023

Related U.S. Application Data
(60) Provisional application No. 63/302,441, filed on Jan. 24, 2022.

(51) Int. Cl. A61M 25/06

(52) U.S. Cl. CPC A61M 25/0631

(57) ABSTRACT

A catheter system may include a blood shield secured within a catheter hub. The blood shield may include a distal end, a proximal end, a blood shield lumen extending through the distal end and the proximal end, and an inner wall forming the blood shield lumen. The inner wall may include a narrowed inner diameter portion or a shield bump. The catheter system may include an introducer needle having a sharp distal tip and a needle bump. The introducer needle may extend through the catheter and the blood shield. In response to proximal retraction of the introducer needle, the needle bump may be configured to contact the narrowed inner diameter portion or the shield bump to pull the blood shield proximally out of the catheter hub. The sharp distal tip may be disposed within the blood shield when the needle bump contacts the narrowed inner diameter portion or the shield bump.

### RELATED APPLICATIONS

This application claims the benefit of U.S. Provisional Patent Application No. 63/302,441, filed Jan. 24, 2022, and entitled CATHETER SYSTEM HAVING A BLOOD SHIELD, which is incorporated herein in its entirety.

### BACKGROUND

A common type of catheter system includes a peripheral intravenous catheter (“PIVC”) that is over-the-needle. As its name implies, the PIVC that is over-the-needle may be mounted over an introducer needle having a sharp distal tip. The catheter system may include a catheter hub, the PIVC extending distally from the catheter hub, and the introducer needle extending through the PIVC. The PIVC and the introducer needle may be assembled such that the distal tip of the introducer needle extends beyond the distal tip of the PIVC with the bevel of the needle facing up away from skin of the patient immediately prior to insertion into the skin. The PIVC and the introducer needle are generally inserted at a shallow angle through the skin into vasculature of the patient.

In order to verify proper placement of the introducer needle and/or the PIVC in the blood vessel, a clinician may confirm that there is flashback of blood in a flashback chamber of the catheter system. In some instances, blood may travel into the introducer needle and then out of a flashback notch in the introducer needle to reach the flashback chamber, where the blood is visible to the clinician. Once placement of the introducer needle has been confirmed by observation of the blood, the clinician may further advance the catheter along the introducer needle, creating a space between a catheter hub and a needle housing. The clinician may remove the introducer needle from the catheter system, leaving the PIVC in place in the blood vessel for future blood withdrawal or fluid infusion. When the introducer needle is removed from the catheter system, blood from the flashback notch or the sharp distal tip may splatter due to the space between the catheter hub and the needle housing. The splatter of blood endangers the clinician and/or the patient.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to vascular access devices. More particularly, in some embodiments, the present disclosure relates to catheter assemblies configured to shield an introducer needle. In first set of embodiments, a catheter system may include a catheter hub, which may include a distal end, a proximal end, and a catheter hub lumen extending through the distal end of the catheter hub and the proximal end of the catheter hub. A catheter may extend distally from the distal end of the catheter hub.

The catheter system may include a blood shield secured within the catheter hub lumen. The blood shield may include a distal end, a proximal end, a blood shield lumen extending through the distal end of the blood shield and the proximal end of the blood shield, and an inner wall forming the blood shield lumen. The inner wall may include a shield bump, which may be annular.

The catheter system may include an introducer needle, which may include a sharp distal tip and a needle bump. The introducer needle may extend through the catheter and the blood shield. In response to proximal retraction of the introducer needle, the needle bump may be configured to contact the shield bump to pull the blood shield proximally out of the proximal end of the catheter hub. The sharp distal tip may be disposed within the blood shield when the needle bump contacts the shield bump. The introducer needle may include a notch proximal to the needle bump. The notch may be disposed within the blood shield when the needle bump contacts the shield bump.

The catheter system may include a septum disposed within the catheter hub lumen. The catheter system may include a septum actuator disposed within the catheter hub lumen and configured to open the septum. At least a portion of the blood shield may be disposed within the septum actuator. The distal end of the blood shield may be disposed in an interference fit with an inner surface of the septum actuator. The distal end of the blood shield may be tipped.

The catheter system may include a housing, which may include a barrel. The catheter system may include a needle hub coupled the introducer needle and movably disposed within the barrel. The catheter system may include a spring disposed within the housing. The catheter system may include a push button. In response to depression of the push button, the spring may be configured to expand proximally and move the needle hub proximally within the barrel to retract the introducer needle proximally.

A portion of the needle hub may be disposed within the catheter hub. A proximal end of the blood shield may be disposed within the portion of the needle hub. The inner wall of the blood shield may include a distal cylindrical shape distal to the shield bump and a proximal cylindrical shape proximal to the shield bump. The sharp distal tip may be disposed within the proximal cylindrical shape when the needle bump contacts the shield bump. The introducer needle may include a notch proximal to the needle bump. The notch may be disposed within the proximal cylindrical shape when the needle bump contacts the shield bump.

In a second set of embodiments, a catheter system may include a catheter hub, which may include a distal end, a proximal end, and a catheter hub lumen extending through the distal end of the catheter hub and the proximal end of the catheter hub. The catheter system may include a catheter extending distally from the distal end of the catheter. The catheter system may include a blood shield secured within the catheter hub lumen. The blood shield may include a distal end, a proximal end, a blood shield lumen extending through the distal end of the blood shield and the proximal end of the blood shield, and an inner wall forming the blood shield lumen. The inner wall may include a narrowed inner diameter portion.

The catheter system may include a septum disposed within the proximal end of the blood shield. The septum may be in an interference fit with the inner wall of the blood shield to secure the septum within the blood shield lumen. The catheter system may include an introducer needle, which may include a sharp distal tip and a needle bump. The introducer needle may extend through the catheter and the blood shield. In response to proximal retraction of the introducer needle, the needle bump may be configured to contact the narrowed inner diameter portion to pull the blood shield proximally out of the proximal end of the catheter hub. The sharp distal tip may be disposed within the blood shield when the needle bump contacts the narrowed inner diameter portion.

The septum may include multiple air vents extending through the septum and spaced apart from an outer edge of the septum. Air may be configured to travel proximally through the introducer needle, the air vents, and the blood shield. The septum may include a slit configured to receive the introducer needle therethrough. The air vents may be arranged in a symmetric pattern, and the slit may be disposed in a center of the symmetric pattern.

An outer edge of the septum may include air vents to allow air to pass between the septum and the inner wall of the catheter hub. The inner wall of the blood shield may include an annular protrusion. An outer surface of the septum may include an annular recess. The annular protrusion may be disposed within the annular recess.

The narrowed inner diameter portion may include a shield bump. The proximal end of the blood shield may include an annular flange. The annular flange may include multiple air vents configured to allow air to pass between the septum and the inner wall of the catheter hub. The inner wall of the blood shield may include an annular protrusion. The annular protrusion may include multiple vent slots configured to allow air to pass through the blood shield.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1 is an upper perspective view of a prior art catheter system;

FIG. 2 is another upper perspective view of the prior art catheter system;

FIG. 3 is a partial cutaway view of an example catheter hub, according to some embodiments;

FIG. 4 is an upper perspective view of an example catheter system that includes the catheter hub, according to some embodiments;

FIG. 5A is an upper perspective view of an example blood shield, according to some embodiments;

FIG. 5B is a distal end view of the blood shield of FIG. 5A, according to some embodiments;

FIG. 5C is a cross-sectional view of the blood shield of FIG. 5A, according to some embodiments;

FIG. 6 is a cross-sectional view of the catheter hub, according to some embodiments;

FIG. 7 is an enlarged cross-sectional view an example distal end of the blood shield of FIG. 5A in an interference fit with an example septum actuator, according to some embodiments;

FIG. 8A is an upper perspective view of an example introducer needle, according to some embodiments;

FIG. 8B is a cross-sectional view of the introducer needle inside the blood shield of FIG. 5A and an example needle bump contacting an example shield bump, according to some embodiments;

FIG. 8C is a cross-sectional view of the introducer needle disposed within an example catheter when the catheter system of FIG. 4 is in a ready position, according to some embodiments;

FIG. 9 is a cross-sectional view of the catheter system of FIG. 4 in the ready position, according to some embodiments;

FIG. 10 is a cross-sectional view of the catheter system of FIG. 4, illustrating the catheter hub advanced distally along the introducer needle, according to some embodiments;

FIG. 11 is a cross-sectional view of the catheter system of FIG. 4, illustrating the introducer needle partially retracted, according to some embodiments;

FIG. 12 is a cross-sectional view of the catheter system of FIG. 4, illustrating the introducer needle further retracted from FIG. 11 to a position in which the needle bump contacts the shield bump, according to some embodiments;

FIG. 13 is a cross-sectional view of the catheter system of FIG. 4, illustrating the introducer needle further retracted from FIG. 12, according to some embodiments;

FIG. 14 is an upper perspective view of the blood shield and the introducer needle in a shielded position in which the needle bump contacts the shield bump, according to some embodiments;

FIG. 15 is a cross-sectional view of the catheter system of FIG. 4, illustrating the introducer needle further retracted from FIG. 13, according to some embodiments;

FIG. 16 is a cross-sectional view of an example mold that may be used to form the blood shield of FIG. 5A, according to some embodiments;

FIG. 17A is an upper perspective view of another example blood shield, illustrating the introducer needle therein, according to some embodiments;

FIG. 17B is another upper perspective view of the blood shield of FIG. 17A, illustrating the introducer needle therein, according to some embodiments;

FIG. 17C is a cross-sectional view of the blood shield of FIG. 17A, according to some embodiments;

FIG. 17D is a distal end view of an example septum according to some embodiments;

FIG. 17E is an upper perspective view of the blood shield of FIG. 17A with the septum removed for illustrative purposes, according to some embodiments;

FIG. 17F is a cross-sectional view of an example distal end of the blood shield of FIG. 17A with the septum removed for illustrative purposes, according to some embodiments;

FIG. 17G is another upper perspective view of the blood shield device of FIG. 17A, illustrating an example air vents, according to some embodiments;

FIG. 18A is a cross-sectional view of an example catheter system that includes the blood shield of FIG. 17A, according to some embodiments;

FIG. 18B is a cross-sectional view of the catheter system of FIG. 18A, according to some embodiments;

FIG. 19A is a cross-sectional view of the catheter system of FIG. 18A, according to some embodiments;

FIG. 19B is a cross-sectional view of the catheter system of FIG. 18A, according to some embodiments;

FIG. 19C is a cross-sectional view of the catheter system of FIG. 18A, according to some embodiments;

FIG. 20A is an upper perspective view of another example blood shield, according to some embodiments;

FIG. 20B is a cross-sectional view of the blood shield of FIG. 20A, according to some embodiments;

FIG. 21A is an upper perspective view of another example blood shield, according to some embodiments; and

FIG. 21B is a cross-sectional view of the blood shield of FIG. 20A, according to some embodiments.

### DESCRIPTION OF EMBODIMENTS

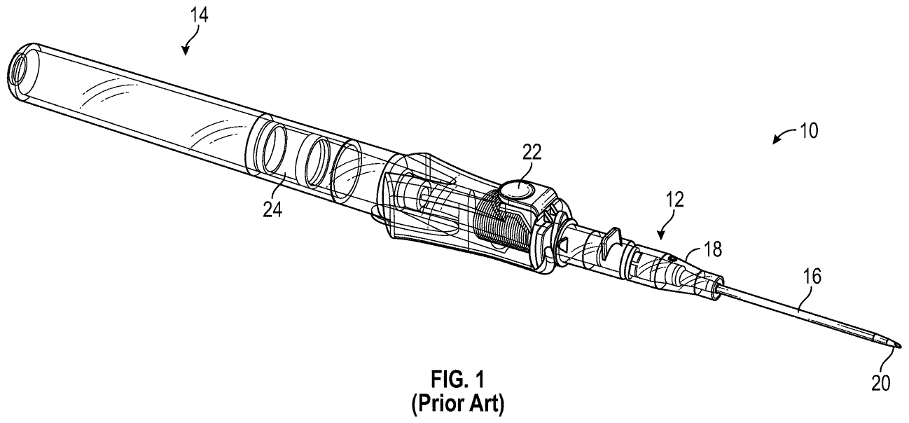

Referring now to FIGS. 1A-1B, a prior art catheter system 10 is illustrated. The prior art catheter system 10 includes a catheter assembly 12 coupled to a needle assembly 14. The catheter assembly 12 includes a catheter 16 extending distally from a distal end of a catheter hub 18. The needle assembly 14 includes an introducer needle 20 extending through the catheter 16 to facilitate insertion of the catheter 16 through a vein of a patient.

The needle assembly 14 includes a push button 22 configured to retract the introducer needle 20 into a barrel 24 of the needle assembly 14 when the push button 22 is pushed or depressed. Some examples of catheter assemblies that may include the push button 22 are the BD INSYTE™ AUTOGUARD™ Shielded IV Catheter, the BD INSYTE™ AUTOGUARD™ BC Shielded IV Catheter, and the AccuCath ACE™ Intravascular Catheter, all available from Becton Dickinson & Company of Franklin Lakes, N.J. Another example of a catheter assembly that may include the push button 22 is the SUPERCATH™ 5 Safety IV Catheter, available from ICU Medical of San Clemente, Calif.

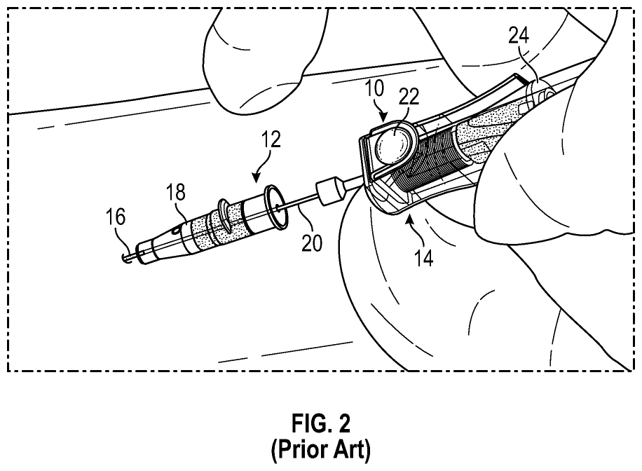

The push button 22 and retraction of the introducer needle 20 help reduce needle stick injuries. In some instances, after the catheter 16 is in the vein and flashback of blood is observed, the catheter 16 may be advanced distally with respect to the introducer needle 20 and further into the vein, creating a space between the catheter hub 18 and the needle assembly 14, as illustrated in FIG. 2. Unfortunately, in some cases, when the push button 22 is activated by a clinician with the catheter hub 18 and the needle assembly 14 spaced apart, blood from the introducer needle 20 may splatter due to inertia, endangering the clinician and/or the patient.

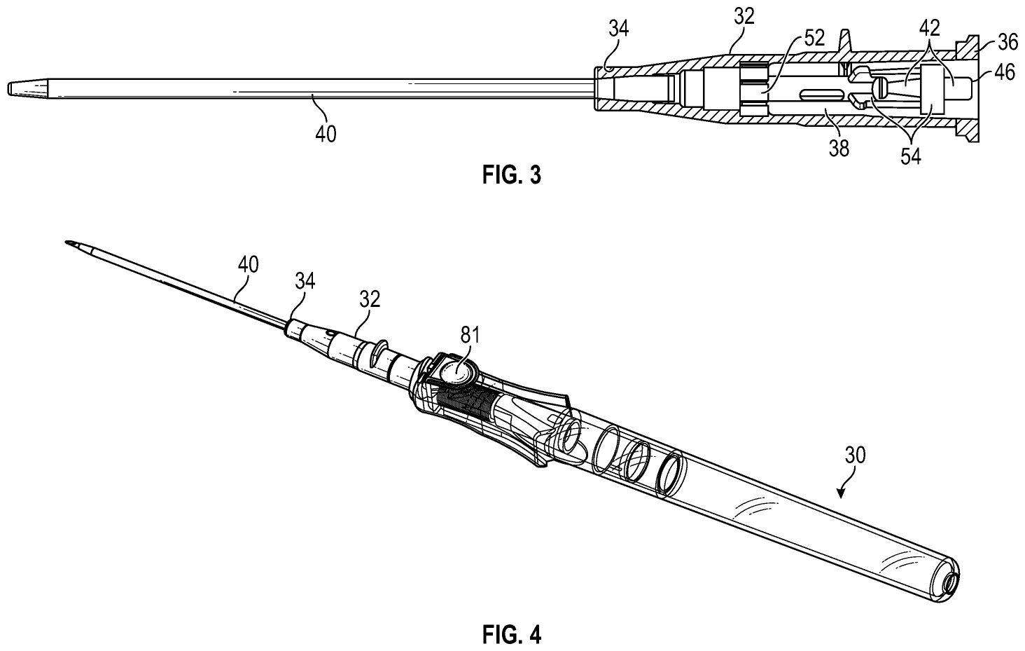

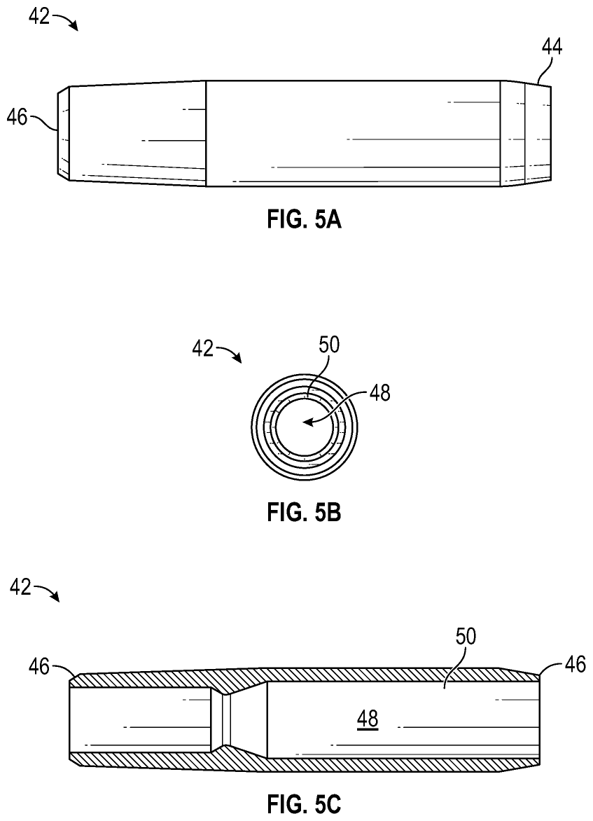

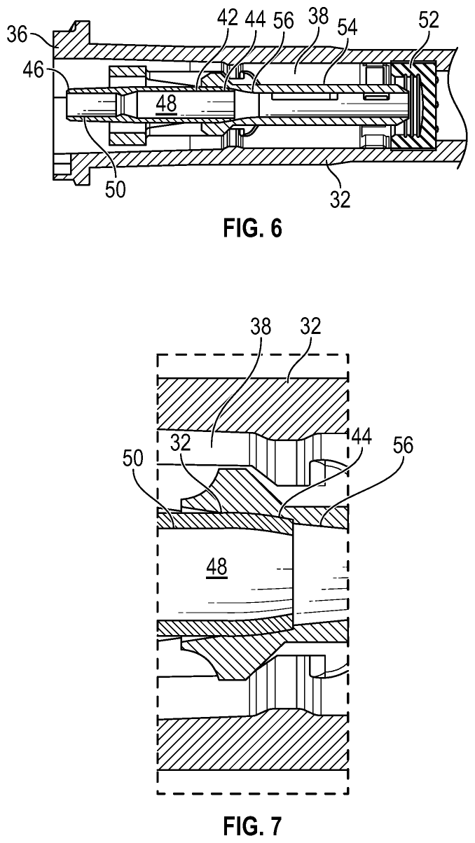

Referring now to FIGS. 3-8, in some embodiments, a catheter system 30 may reduce a risk of splattering or release of blood towards the clinician. In some embodiments, the catheter system 30 may include a catheter hub 32, which may include a distal end 34, a proximal end 36, and a catheter hub lumen 38 extending through the distal end 34 of the catheter hub 32 and the proximal end 36 of the catheter hub 32. In some embodiments, a catheter 40 may extend distally from the distal end of the catheter hub 32. In some embodiments, the catheter 40 may include a peripheral intravenous catheter, a midline catheter, or a peripherally-inserted central catheter.

In some embodiments, the catheter system 30 may include a blood shield 42 secured within the catheter hub lumen 38. In some embodiments, the blood shield 42 may include a distal end 44, a proximal end 46, a blood shield lumen 48 extending through the distal end 44 of the blood shield 42 and the proximal end 46 of the blood shield 42, and an inner wall 50 forming the blood shield lumen 48.

In some embodiments, the catheter system 30 may include a septum 52 disposed within the catheter hub lumen 38. In some embodiments, the catheter system 30 may include a septum actuator 54 disposed within the catheter hub lumen 38 and configured to open the septum 52. In some embodiments, the septum 52 may be in an interference fit with the inner wall 50 of the catheter hub 32 to secure the septum 52 within the catheter hub lumen 38. In some embodiments, after the blood shield 42 is removed from the catheter hub 18, a male luer may be inserted into the proximal end 36 of the catheter hub 18. In some embodiments, the male luer may contact the septum actuator 54 to move the septum actuator 54 distally and through the septum 52.

In some embodiments, at least a portion of the blood shield 42 may be disposed within the septum actuator 54. In some embodiments, the distal end 44 of the blood shield 42 may be disposed in an interference fit with an inner surface 56 of the septum actuator 54, as illustrated, for example, in FIG. 7. In further detail, in some embodiments, an outer diameter of the distal end 44 of the blood shield 42 may be slightly larger than an inner diameter of the septum actuator 54 such that the distal end 44 of the blood shield 42 snugly fits within the septum actuator 54. In some embodiments, the interference fit between the inner surface 56 of the septum actuator 54 and the distal end 44 of the blood shield 42 may secure the blood shield 42 within the catheter hub lumen 38. In some embodiments, the distal end 44 of the blood shield 42 may be tipped, which may match an inner profile of the septum actuator 54 and facilitate securement of the distal end 44 within the septum actuator 54.

In some embodiments, the inner wall 50 may include a shield bump 58, which may include a protrusion. In some embodiments, the blood shield 42 may be barrel shaped or generally cylindrical. In some embodiments, the inner wall 50 of the blood shield 42 further may include a distal cylindrical shape 60 distal to the shield bump 58 and a proximal cylindrical shape 62 proximal to the shield bump 58.

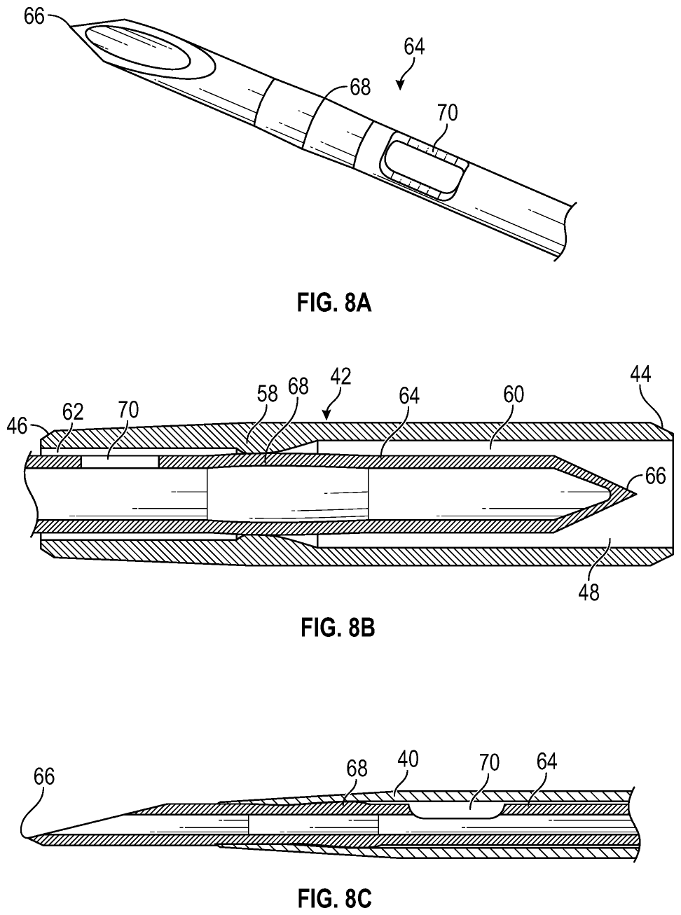

As illustrated in FIG. 8C, in some embodiments, the catheter system 30 may include an introducer needle 64, which may include a sharp distal tip 66 and a needle bump 68. In some embodiments, the introducer needle 64 may extend through the catheter 40 and the blood shield 42 when the catheter system 30 is in a ready position, ready for insertion into the vein of the patient. As illustrated in FIG. 8B, in some embodiments, in response to proximal retraction of the introducer needle 64, the needle bump 68 may be configured to contact the shield bump 58 to pull the blood shield 42 proximally out of the proximal end 36 of the catheter hub 32. In some embodiments, the sharp distal tip 66 may be disposed within the blood shield 42 when the needle bump 68 contacts the shield bump 58.

In some embodiments, the introducer needle 64 may include a notch 70 proximal to the needle bump 68. In some embodiments, the notch 70 may include a flashback notch providing a fluid path from an internal lumen of the needle to an exterior of the introducer needle 64 and disposed within the catheter 40 when the catheter system is in the ready position. In some embodiments, the notch 70 may be disposed within the blood shield 42 when the needle bump 68 contacts the shield bump 58. In some embodiments, the shield bump 58 and/or the needle bump 68 may be annular, which may facilitate catching of the needle bump 68 on the shield bump 58 as the introducer needle 64 is retracted proximally. In some embodiments, the needle bump 68 may be elliptical, and the shield bump 58 may be annular to facilitate interference with the needle bump 68.

In some embodiments, the sharp distal tip 66 may be disposed within the proximal cylindrical shape 62 when the needle bump 68 contacts the shield bump 58. In some embodiments, the notch 70 may be disposed within the proximal cylindrical shape 62 when the needle bump 68 contacts the shield bump 58. Thus, in some embodiments, both the sharp distal tip 66 and the notch 70 may be shielded within and covered by the blood shield 42, which may protect the clinician from blood splatter exiting the sharp distal tip 66 and/or the notch 70.

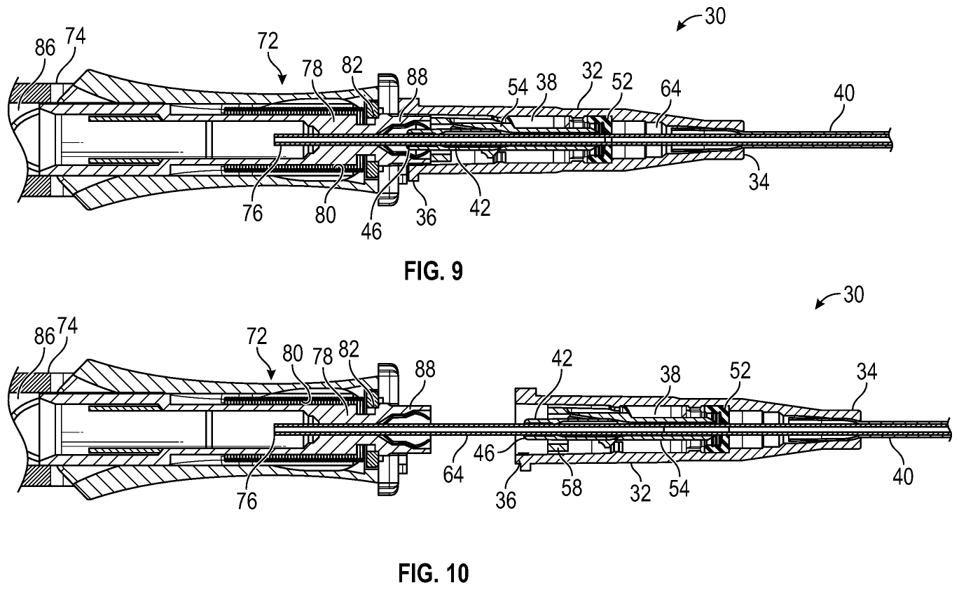

Referring now to FIGS. 9-10, in some embodiments, the catheter system 30 may include a needle assembly, which may include a housing 72. In some embodiments, the housing 72 may include a barrel 74. In some embodiments, the needle assembly may include the introducer needle 64, which may include a sharp distal tip 66 and a proximal end 76. In some embodiments, the needle assembly may include a needle hub 78 coupled to the introducer needle 64 and movably disposed within the barrel 74, which may shield the user from the introducer needle 64 and/or blood after retraction of the introducer needle 64 into the barrel 74. In some embodiments, the needle assembly may include a spring 80 disposed within the housing 72. In some embodiments, the needle assembly may include a push button 81.

In some embodiments, the push button 81 may operate similar to the activation latch described in U.S. Pat. No. 5,501,675, filed Dec. 27, 1994, entitled “SAFETY CATHETER SYSTEM HAVING SAFETY STOP PUSH BUTTON”, which is hereby incorporated by reference in its entirety. In some embodiments, the push button 81 may operate in another manner to shield a particular introducer needle. In some embodiments, the spring 80 may be located about introducer needle 64, and the spring 80 may extend between the needle hub 78 and a distal end of barrel 74. In some embodiments, the push button 81 may extend into the barrel 74 via a cavity or slot formed in the barrel 74 adjacent to the distal end of the barrel 74. In some embodiments, the push button 81 may include an opening 82 configured to allow the introducer needle 64 and the needle hub 78 to extend through the push button 81. In some embodiments, the opening 82 may be keyhole shaped. In some embodiments, the push button 81 may include a projection that may extend toward the distal end of catheter 40 and the catheter system 30.

In some embodiments, when push button 81 is “up” in a non-activated position, a smaller portion of the opening 82 may be in communication with a lumen 86 of the barrel 74. In some embodiments, in this position, the smaller portion of the opening 82 may engage the needle hub 78 and hold the needle hub 78 adjacent to the distal end of barrel 74 against the force of the spring 80. In some embodiments, the needle hub 78 may include a distal flare so that a medial portion of the needle hub 78 has a smaller diameter than the distal flare. In some embodiments, the needle hub 78 may include an hourglass shape. In some embodiments, the distal flare and/or the hourglass shape may facilitate engagement between the smaller portion of the opening 82 and the needle hub 78. In some embodiments, when the push button 81 is in the non-activated position, the projection may be located inside the catheter hub 32. Thus, in some embodiments, when the catheter 40 is still located on the introducer needle 64 with the catheter hub 32 adjacent to the distal end of the housing 72, the projection may prevent the push button 81 from being depressed or moved “down” into the activated position.

In some embodiments, when the catheter 40 is moved off or along the introducer needle 64 so the catheter hub 32 is not adjacent to the distal end of housing 72, the push button 81 can be moved “down,” i.e., activated, because the catheter hub 32 no longer interferes with the movement of the projection. In some embodiments, in this position, a larger portion of the opening 82 may no longer engage the needle hub 78. In some embodiments, the larger portion of the opening 82 should be larger than the maximum diameter of the needle hub 78. In some embodiments, the spring 80 can thus force the needle hub 78 to a proximal end of the barrel 74 and withdraw the sharp distal tip 66 of the introducer needle 64 into the barrel 74.

In some embodiments, a portion 88 of the needle hub 78 may be disposed within the catheter hub 32 when the catheter system 30 is in the ready position, as illustrated in FIG. 9. In some embodiments, the proximal end 46 of the blood shield 42 may be disposed within the portion 88 of the needle hub 78 when the catheter system 30 is in the ready position, which may facilitate stability or securement of the blood shield 42. The above description of the needle assembly is just one example of a needle assembly with which the blood shield 42 may be used.

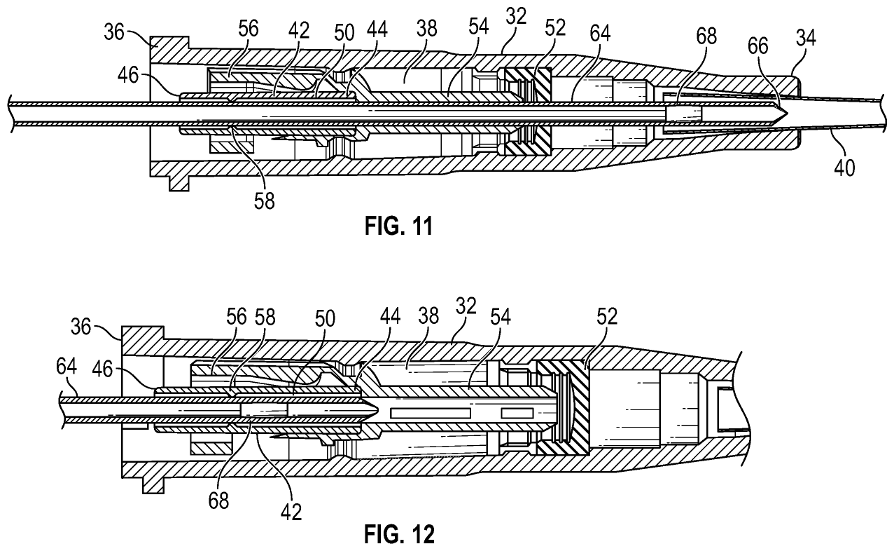

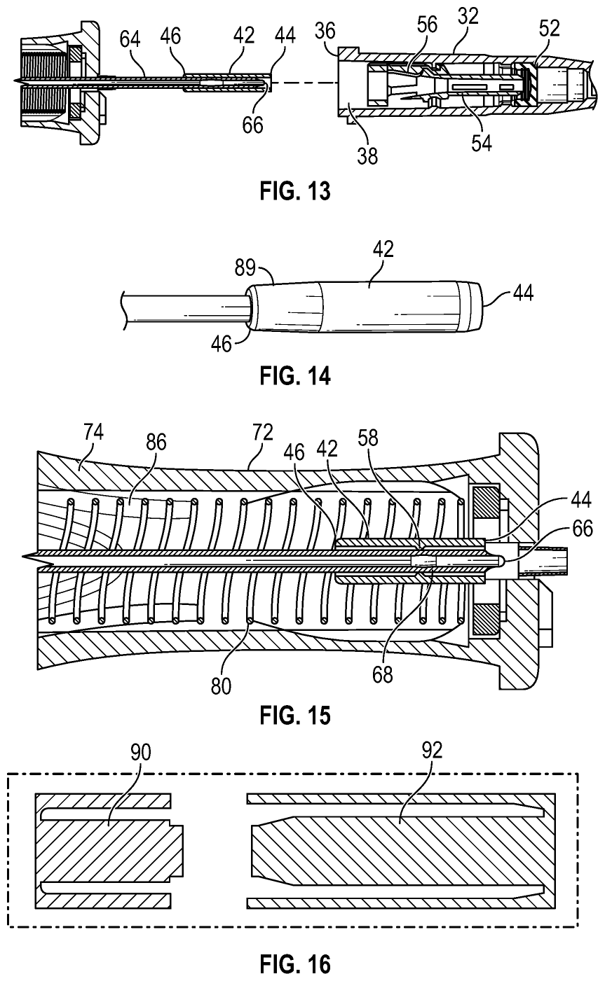

Referring now to FIG. 11, the introducer needle 64 is illustrated moving proximally from the ready position, according to some embodiments. Referring now to FIG. 12, the introducer needle 64 is illustrated moved further proximally than FIG. 11 such that the needle bump 68 contacts the shield bump 58, according to some embodiments. Referring now to FIG. 13, according to some embodiments, the introducer needle 64 is illustrated moved further proximally than FIG. 13 such that the introducer needle 64 pulls the blood shield 42 proximally from the catheter hub 32, where the blood shield 42 was in a secured position. In some embodiments, the needle bump 68 contacting and interfering with the shield bump 58 facilitates proximal movement of the blood shield 42 from the secured position.

Referring now to FIG. 14, the sharp distal tip 66 is shielded within the blood shield 42 when the needle bump 68 contacts the shield bump 58, according to some embodiments. Referring now to FIG. 15, the blood shield 42 and the introducer needle 64 are illustrated shielded within the housing 72. In some embodiments, in response to depression of the push button, the spring 80 may be configured to expand proximally as illustrated, for example, in FIG. 15, and move the needle hub 78 proximally within the barrel 74 to retract the introducer needle 64 and the blood shield 42 proximally.

Referring back to FIG. 14, in some embodiments, the proximal end 46 of the blood shield 42 may include a conical taper feature 89 to guide the blood shield 42 into the housing 72 during proximal retraction of the introducer needle 64. In these embodiments, the conical taper feature 89 may taper inwardly in a proximal direction. In some embodiments, an outer surface of the distal end 44 and/or an outer surface of the proximal end 46 may be stepped or chamfered, which may reduce an outer diameter of the blood shield 42. In some embodiments, the blood shield 42 may be constructed of polypropylene, such as PH592 or RP142, or another suitable material.

Referring now to FIG. 16, a first half 90 of a mold to mold the proximal end 46 of the blood shield 42 is illustrated, according to some embodiments. In some embodiments, a second half 92 of the mold to mold the distal end 44 of the blood shield 42 is illustrated, according to some embodiments. In some embodiments, both of the first half 90 and the second half 92 may include a cutout portion to form a portion of the shield bump 58. In some embodiments, the blood shield 42 may be monolithically formed as a single unit and may include a single molded piece.

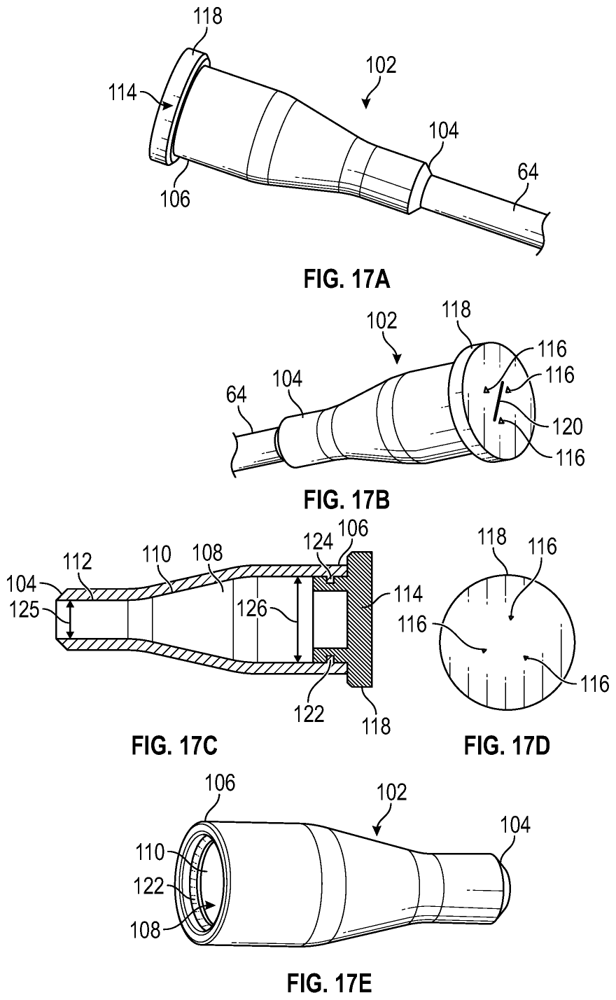

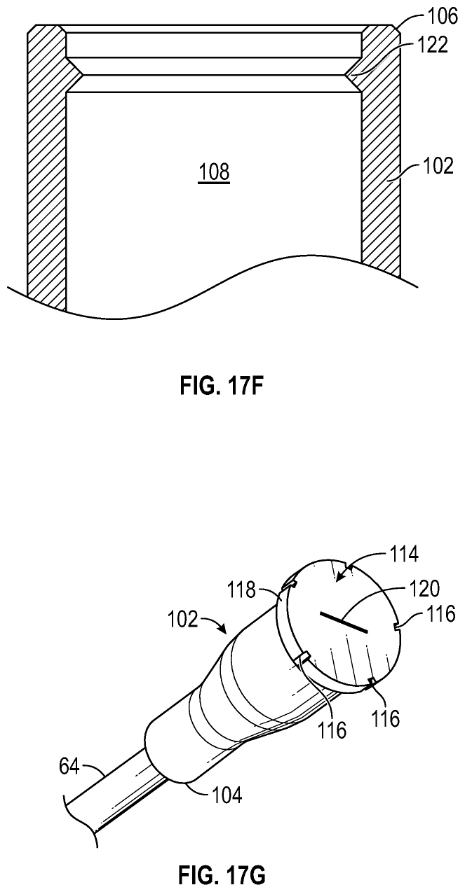

Referring now to FIGS. 17A-19C, a catheter system 100 is illustrated, according to some embodiments. In some embodiments, the catheter system 100 may be similar or identical to the catheter system 30 in terms of one or more components and/or operation. In some embodiments, the catheter system 100 may include a catheter hub 32, which may include a distal end, a proximal end 36, and a catheter hub lumen 38 extending through the distal end of the catheter hub and the proximal end 36 of the catheter hub 32. In some embodiments, the catheter system 100 may include the catheter 40 extending distally from the distal end of the catheter hub 32.

In some embodiments, the catheter system 100 may include a blood shield 102 secured within the catheter hub lumen 38. In some embodiments, the blood shield 102 may be monolithically formed as a single unit and may include a single molded piece. In some embodiments, the blood shield 102 may be similar or identical to the blood shield 42 of FIGS. 3-16 in terms of one or more components and/or operation. In some embodiments, the blood shield 102 may include a distal end 104, a proximal end 106, a blood shield lumen 108 extending through the distal end 104 of the blood shield 102 and the proximal end 106 of the blood shield 102, and an inner wall 110 forming the blood shield lumen 108.

In some embodiments, the inner wall 110 may include a narrowed inner diameter portion 112. In some embodiments, the catheter system 100 may include a septum 114 disposed within the proximal end 106 of the blood shield 102. In some embodiments, the septum 114 may be in an interference fit with the inner wall 110 of the blood shield 102 to secure the septum 114 within the blood shield lumen 108. In some embodiments, the septum 114 may prevent blood leakage from the distal end 104 of the blood shield 102 and may help avoid blood exposure.

In some embodiments, the catheter system 100 may include an introducer needle 64, which may include a sharp distal tip 66 and a needle bump 68. In some embodiments, the introducer needle 64 may extend through the catheter 40 and the blood shield 102 when the catheter system 100 is in a ready position, ready for insertion into the vein of the patient. In some embodiments, in response to proximal retraction of the introducer needle 64, the needle bump 68 may be configured to contact the narrowed inner diameter portion 112 to pull the blood shield 102 proximally out of the proximal end 36 of the catheter hub 32.

In some embodiments, the sharp distal tip 66 may be disposed within the blood shield 102 when the needle bump 68 contacts the narrowed inner diameter portion 112. In some embodiments, both the sharp distal tip 66 and a notch (such as the notch 70 of FIGS. 8A-8C) may be shielded within the blood shield 42, which may protect the clinician from blood splatter exiting the sharp distal tip 66 and/or the notch.

In some embodiments, the septum 114 may include multiple air vents 116 extending through the septum 114 and spaced apart from an outer edge 118 of the septum 114. In some embodiments, the air vents 116 may facilitate blood flashback entering into the catheter system 100 when the introducer needle 64 and/or the catheter 40 is inserted into the vein of the patient. In some embodiments, air may be configured to travel proximally through the introducer needle 64, the air vents 116, and the blood shield 102. In some embodiments, the air vents 116 may be sized and configured to pass air but not liquid, such as blood. In some embodiments, the septum 114 may include a slit 120 configured to receive the introducer needle 64 therethrough. In some embodiments, the air vents 116 may be arranged in a symmetric pattern, such as a triangle. In some embodiments, the slit 120 may be disposed in a center of the symmetric pattern, as illustrated, for example in FIG. 17B.

As illustrated in FIG. 17G, in some embodiments, the outer edge 118 of the septum 114 may include the air vents 116 to allow air to pass between the septum 114 and the inner wall 50 of the catheter hub 32. In some embodiments, the outer edge 118 may be in an interference fit with the inner wall 50 of the catheter hub 32. In some embodiments, in further detail, a circumference of the outer edge 118 may be slightly larger than a circumference of the catheter hub lumen 38, and the septum 114 may be compressed to secure the septum 114 within the catheter hub 32. In some embodiments, the outer edge may be circular to facilitate alignment in the catheter hub 32.

As illustrated in FIGS. 17C and 17E-17F, in some embodiments, the inner wall 50 of the blood shield 102 may include an annular protrusion 122. In some embodiments, an outer surface of the septum 114 may include an annular recess 124. In some embodiments, the annular protrusion 122 may be disposed within the annular recess 124, which may secure a distal portion of the septum 114 within the blood shield 102.

In some embodiments, the narrowed inner diameter portion 112 may be disposed proximal to a larger diameter 126 at the distal end 104. In some embodiments, the larger diameter 126 may receive the septum 114. In some embodiments, the narrowed inner diameter portion 112 may have a diameter 125 slightly greater than an outer diameter of the introducer needle 64 at the needle bump 68 to allow the introducer needle 64 to slide through the blood shield 42 but limit space out of which blood could leak.

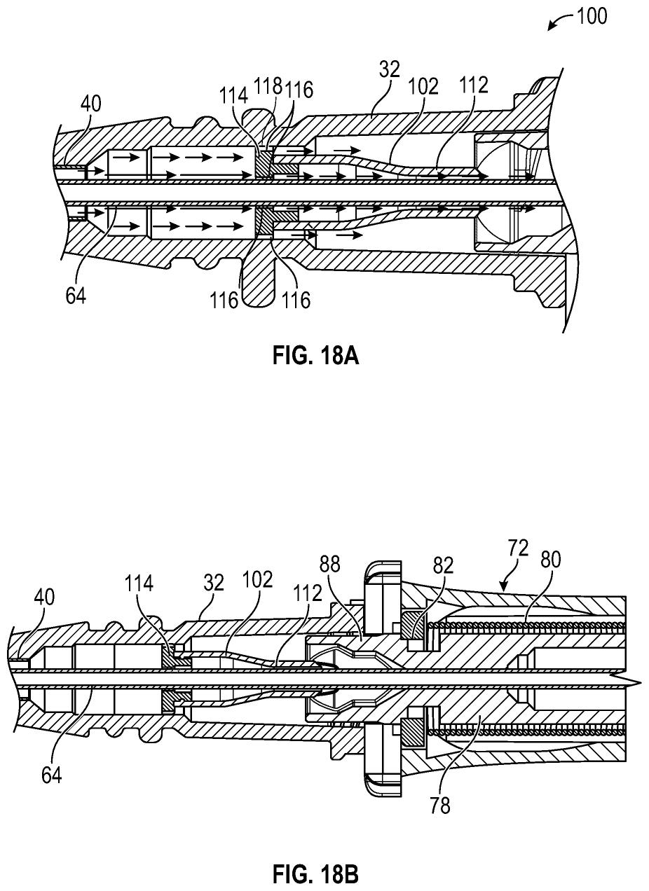

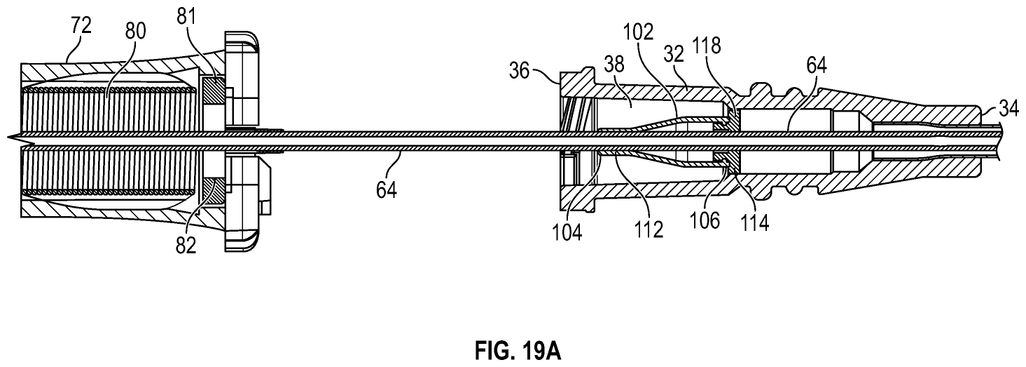

FIG. 18A illustrates passage of air through the air vents 116 in response to blood flashback and entry of the introducer needle 64 and the catheter into the vein of the patient. FIG. 18B is an enlarged cross-sectional view of FIG. 18A. FIGS. 18A-18B illustrate the blood shield 102 secured within the catheter hub 32 due to the interference fit between the septum 114 and the catheter hub 32, according to some embodiments. Although the air vents 116 on both the outer edge of the septum 114 and spaced apart from the outer edge of the septum 114 are illustrated, which may provide greater venting, it is understood that in some embodiments, the septum 114 may include the air vents 116 on the outer edge 118 of the septum 114 or the air vents 116 spaced apart from the outer edge 118 of the septum 114.

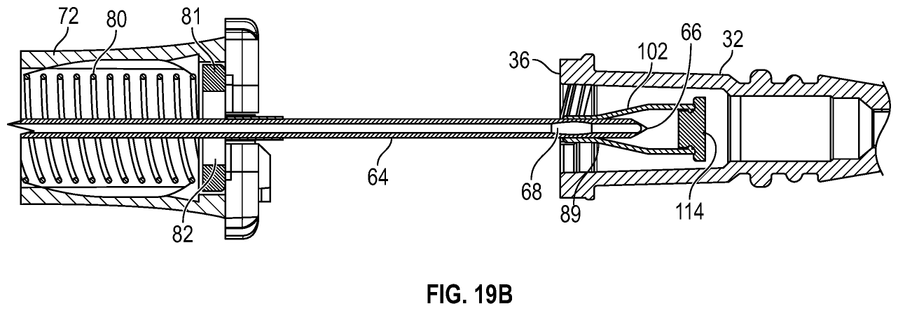

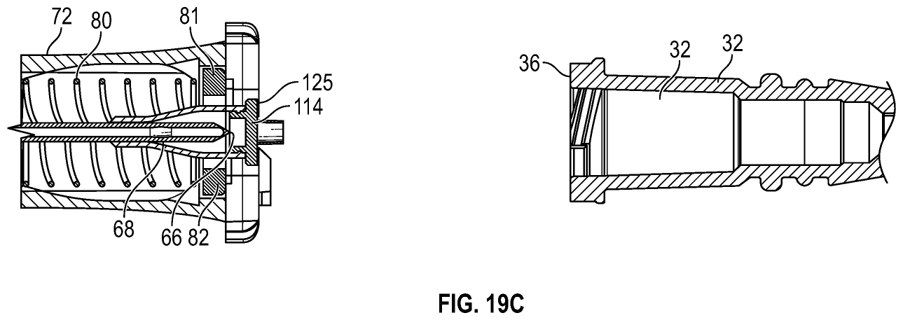

FIG. 19A illustrates the blood shield 102 secured within the catheter hub 32 in an initial or first position. In FIG. 19A, the catheter hub 32 is advanced distally along the introducer needle 64 after the introducer needle 64 is placed within the vein of the patient, creating a space between the catheter hub 32 and the housing 72. In some embodiments, the initial or first position may also correspond to a position of the blood shield 102 when the catheter system 100 is in the ready position.

FIG. 19B illustrates the introducer needle 64 being retracted proximally, following the configuration of FIG. 19A. In some embodiments, in response to proximal retraction of the introducer needle 64, the needle bump 68 may contact the narrowed inner diameter portion 112 to pull the blood shield 102 from the catheter hub 32 and into the housing 72. In some embodiments, as illustrated in FIG. 19C, further retraction of the introducer needle 64 in a proximal direction may move the blood shield 102 into the housing 72. In some embodiments, the septum 114 may seal an opening 125 of the housing 72 through which the blood shield 102 entered the housing 72, which may prevent blood leakage from the housing 72. In some embodiments, the position of the blood shield 102 in FIG. 19C may correspond to a final position of the blood shield 102 when the introducer needle 64 is fully retracted.

In some embodiments, the proximal end 46 of the blood shield 42, or an outer surface of the blood shield aligned with the narrowed inner diameter portion 112, may include a conical taper feature 89 to guide the blood shield 42 into an opening 125 in the housing 72 during proximal retraction of the introducer needle 64. In these embodiments, the conical taper feature 89 may taper inwardly in a proximal direction.

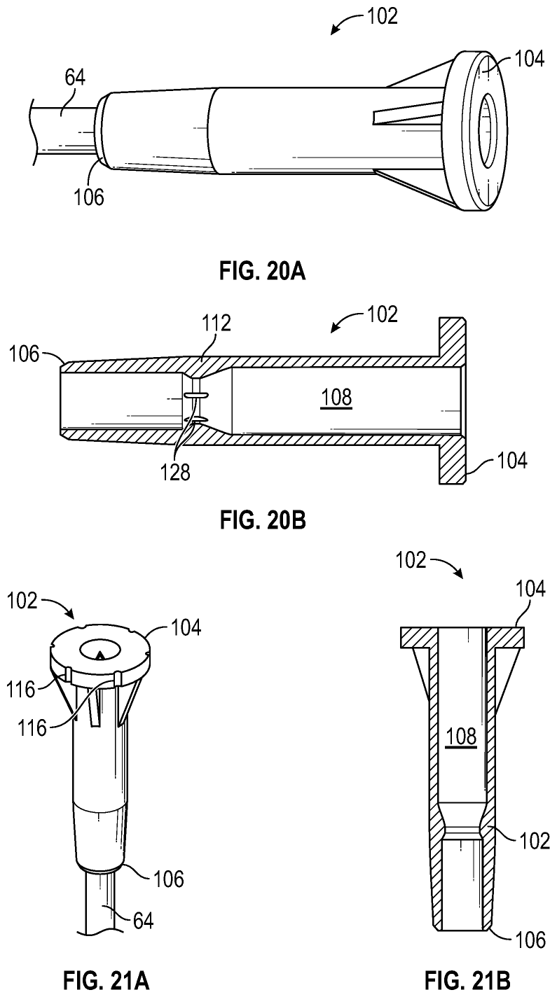

Referring now to FIGS. 20A-20B, a blood shield is illustrated, according to some embodiments. In some embodiments, the blood shield of FIGS. 20A-20B may be similar or identical to the blood shield of FIGS. 17-19 in terms of one or more components and/or operation. In some embodiments, the narrowed inner diameter portion 112 may include a shield bump, similar to the shield bump 58 of FIGS. 3-15, for example.

In some embodiments, the shield bump may include an annular protrusion, as illustrated, for example in FIG. 20B. In some embodiments, the annular protrusion may include multiple vent slots 128 configured to allow air to pass through the blood shield 102 into the catheter hub 32. In some embodiments, the vent slots 128 may be sized and configured to allow air but not liquid, such as blood, to pass. In some embodiments, the vent slots 128 may be arranged generally parallel to a longitudinal axis of the blood shield 102, which may avoid the vent slots 128 from interfering with a function of the narrowed inner diameter portion 112 in preventing passage of the needle bump 68.

In some embodiments, the distal end 104 may include an annular flange. In these embodiments, the distal end 104 may be disc-like. In some embodiments, the outer edge of the distal end 104 may be in an interference fit with the inner wall 50 of the catheter hub 32, similar to the outer edge 118 in FIG. 18A.

Referring now to FIGS. 21A-21B, a blood shield is illustrated, according to some embodiments. In some embodiments, the blood shield of FIGS. 20A-20B may be similar or identical to the blood shield 102 of FIGS. 17-19 and/or the blood shield FIGS. 20A-20B in terms of one or more components and/or operation. In some embodiments, the narrowed inner diameter portion 112 may include a shield bump, similar to the shield bump 58 of FIGS. 3-15, for example. In some embodiments, the air vents 116 may be disposed in an outer edge of the distal end 104 of the blood shield 102. In some embodiments, the distal end 104 may include an annular flange. In these embodiments, the distal end 104 may be disc-like. In some embodiments, the air vents 116 may be disposed in an outer edge of the annular flange. In some embodiments, the air vents 116 may be configured to allow air to pass between the septum 114 and the inner wall 50 of the catheter hub 32. In some embodiments, the outer edge of the distal end 104 may be in an interference fit with the inner wall 50 of the catheter hub 32, similar to the outer edge 118 in FIG. 18A.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. A catheter system, comprising:
a catheter hub, comprising a distal end, a proximal end, and a catheter hub lumen extending through the distal end of the catheter hub and the proximal end of the catheter hub;
a catheter extending distally from the distal end of the catheter hub;
a blood shield secured within the catheter hub lumen, wherein the blood shield comprises a distal end, a proximal end, a blood shield lumen extending through the distal end of the blood shield and the proximal end of the blood shield, and an inner wall forming the blood shield lumen, wherein the inner wall comprises a shield bump; and
an introducer needle comprising a sharp distal tip and a needle bump, wherein the introducer needle extends through the catheter and the blood shield, wherein in response to proximal retraction of the introducer needle, the needle bump is configured to contact the shield bump to pull the blood shield proximally out of the proximal end of the catheter hub, wherein the sharp distal tip is disposed within the blood shield when the needle bump contacts the shield bump.

2. The catheter system of claim 1, wherein the introducer needle comprises a notch proximal to the needle bump, wherein the notch is disposed within the blood shield when the needle bump contacts the shield bump.

3. The catheter system of claim 1, further comprising a septum disposed within the catheter hub lumen, and a septum actuator disposed within the catheter hub lumen and configured to open the septum, wherein at least a portion of the blood shield is disposed within the septum actuator.

4. The catheter system of claim 3, wherein the distal end of the blood shield is disposed in an interference fit with an inner surface of the septum actuator.

5. The catheter system of claim 4, wherein the distal end of the blood shield is tipped.

6. The catheter system of claim 1, wherein the shield bump is annular.

7. The catheter system of claim 1, further comprising:
a housing comprising a barrel;
a needle hub coupled the introducer needle and movably disposed within the barrel;
a spring disposed within the housing; and
a push button, wherein in response to depression of the push button, the spring is configured to expand proximally and move the needle hub proximally within the barrel to retract the introducer needle proximally.

8. The catheter system of claim 7, wherein a portion of the needle hub is disposed within the catheter hub, wherein a proximal end of the blood shield is disposed within the portion of the needle hub.

9. The catheter system of claim 1, wherein the inner wall of the blood shield further comprises a distal cylindrical shape distal to the shield bump and a proximal cylindrical shape proximal to the shield bump.

10. The catheter system of claim 9, wherein the sharp distal tip is disposed within the proximal cylindrical shape when the needle bump contacts the shield bump.

11. The catheter system of claim 9, wherein the introducer needle comprises a notch proximal to the needle bump, wherein the notch is disposed within the proximal cylindrical shape when the needle bump contacts the shield bump.

12. A catheter system, comprising:
a catheter hub, comprising a distal end, a proximal end, and a catheter hub lumen extending through the distal end of the catheter hub and the proximal end of the catheter hub;
a catheter extending distally from the distal end of the catheter hub;
a blood shield secured within the catheter hub lumen, wherein the blood shield comprises a distal end, a proximal end, a blood shield lumen extending through the distal end of the blood shield and the proximal end of the blood shield, and an inner wall forming the blood shield lumen, wherein the inner wall comprises a narrowed inner diameter portion;
a septum disposed within the proximal end of the blood shield;
an introducer needle comprising a sharp distal tip and a needle bump, wherein the introducer needle extends through the catheter and the blood shield, wherein in response to proximal retraction of the introducer needle, the needle bump is configured to contact the narrowed inner diameter portion to pull the blood shield proximally out of the proximal end of the catheter hub, wherein the sharp distal tip is disposed within the blood shield when the needle bump contacts the narrowed inner diameter portion.

13. The catheter system of claim 12, wherein the septum is in an interference fit with the inner wall of the blood shield to secure the septum within the blood shield lumen.

14. The catheter system of claim 12, wherein the septum comprises a plurality of air vents extending through the septum and spaced apart from an outer edge of the septum, wherein air is configured to travel proximally through the introducer needle, the plurality of air vents, and the blood shield.

15. The catheter system of claim 14, wherein the septum further comprises a slit configured to receive the introducer needle therethrough.

16. The catheter system of claim 15, wherein the plurality of air vents are arranged in a symmetric pattern, wherein the slit is disposed in a center of the symmetric pattern.

17. The catheter system of claim 12, wherein an outer edge of the septum comprises a plurality of air vents to allow air to pass between the septum and the inner wall of the catheter hub.

18. The catheter system of claim 12, wherein the inner wall of the blood shield comprises an annular protrusion, wherein an outer surface of the septum comprises an annular recess, wherein the annular protrusion is disposed within the annular recess.

19. The catheter system of claim 12, wherein the narrowed inner diameter portion comprises a shield bump, wherein the proximal end of the blood shield comprises a annular flange, wherein the annular flange comprises a plurality of air vents configured to allow air to pass between the septum and the inner wall of the catheter hub.

20. The catheter system of claim 12, wherein the inner wall of the blood shield comprises an annular protrusion, wherein the annular protrusion comprises a plurality of vent slots configured to allow air to pass through the blood shield.
